(19) United States
(12) Patent Application Publication
(10) Pub. No.: US 2006/0106450 A1
(43) Pub. Date: May 18, 2006

(54) Geometric flow regulator

(75) Inventor: Shmuel Ben Muvhar, D.N. Beit Shean Valley (IL)

Correspondence Address: WOLF, BLOCK, SCHORR & SOLIS-COHEN LLP, 250 PARK AVENUE, NEW YORK, NY, 10177, US

(73) Assignee: Neovasc Medical Ltd., Or-Yehuda (IL)

(21) Appl. No.: 10/524,077

(22) PCT Filed: Apr. 10, 2003
(86) PCT No.: PCT/IL03/00303

§ 371 Date: Sep. 21, 2005

(30) Foreign Application Priority Data
Aug. 8, 2002 (IL) 151162
Sep. 25, 2002 (IL) 151931
Sep. 26, 2002 (US) 10/239980
Oct. 3, 2002 (WO) PCT/IL02/00805
Oct. 17, 2002 (IL) 152366
Dec. 30, 2002 (IL) 153753

(51) Int. Cl. A61F 2/01; A61F 2/06

(52) U.S. Cl. 623/1.15; 606/194; 606/200; 623/1.24

(57) ABSTRACT

A tubular implant for obstructing blood flow through a blood vessel, (110) the implant comprising an outer surface having a geometry of a tube, at least a portion of which is adapted for contacting a blood vessel and an inner surface defining a passage through which blood flows, wherein the distance between the inner surface and the outer surface is non-uniform along an axis of said tube.

### RELATED APPLICATIONS

This application claims priority from PCT/IL02/00805 filed Oct. 3, 2002, which is a CIP of PCT/IL01/00284 filed Mar. 27, 2001, now U.S. Ser. No. 10/239,980 which is a CIP of U.S. Ser. No. 09/534,968, the disclosure of all of which are incorporated herein by reference.

This application also claims priority from the following applications: Israel Application No. 151162, filed on Aug. 8, 2002, Israel Application No. 151931, filed on Sep. 25, 2002, U.S. application Ser. No. 10/239,980, filed on Sep. 26, 2002, PCT Application No. PCT/IL02/00805, filed on Oct. 3, 2002, Israel Application No. 152366, filed on Oct. 17, 2002 and Israel Application No. 153753, filed on Dec. 30, 2002. The disclosure of all of which are also incorporated herein by reference.

### FIELD OF THE INVENTION

The present invention relates to devices for partially obstructing blood flow through a blood vessel.

### BACKGROUND OF TH INVENTION

Angiogenesis, is a process by which new arteries are created within tissue to bypass occluded vessels or areas of poor circulation. Angiogenesis does not usually occur to any great degree naturally and various procedures have been suggested to encourage it, particularly in the heart. For example, in coronary tissue, Trans-Myocardial Revascularization (TMR) is a process in which multiple holes are drilled in the heart with the intent of causing new vessels to form.

Constriction of the coronary sinus to reduce the flow of venous blood that passes through it to the right atrium has been shown to promote angiogenesis. (See: “The Surgical Management of Coronary Artery Disease: Background, Rationale, Clinical Experience” by C. S. Beck and B. L. Brofman, American College of Physicians in Annals of Internal Medicine; Vol. 45, No. 6, December 1956.)

Ruiz in U.S. Pat. No. 6,120,534 teaches a stent having a crimped flow passage for temporary reduction of blood flow in a pulmonary artery of a newborn.

Palmaz in U.S. Pat. No. 5,382,261 teaches a stent having a hollowed, bullet-shaped portion that fully occludes blood flow and promotes clot formation within the hollowed portion.

Mobin-Uddin in U.S. Pat. No. 4,727,873 teaches an embolus trap that anchors in a blood vessel with wires of uniform thickness.

Carpentier et al. in U.S. Pat. No. 4,106,129, Pavcnik et al. in U.S. Pat. No. 5,397,351 and Bailey et al. in US Patent application 2001/0021872 teach wires of uniform thickness that anchor a valve in the heart.

Block et al. in U.S. Pat. No. 5,554,185 teach an inflatable cardiac valve.

Khosravi, in U.S. Pat. No. 5,925,063 teaches multiple overlapping flaps that may be configured into a valve, blood filter, blood flow occluding device or flow regulator.

Anderson et al. in U.S. Pat. No. 6,168,614 teach a cardiac valve that is expanded in vivo using a balloon.

The disclosure of all the above-noted prior art is incorporated herein by reference.

### SUMMARY OF THE INVENTION

An aspect of some embodiments of the invention relates to a flow-obstructing implant comprising an outer surface, at least a portion of which is adapted to contact a blood vessel and an inner surface defining a flow passage. In an exemplary embodiment, at least a portion of the walls surrounding the flow passage are thickened so as to decrease the mean cross-sectional diameter, providing increased flow obstruction.

In an exemplary embodiment, at least a portion of the implant comprises materials that expand upon absorbing liquid during in vivo implantation so that, for example, the implant expands to a flow-obstructing configuration following implantation.

Optionally, the implant comprises a material that compresses under pressure for example when a balloon catheter is inflated against it. Upon inflation of the balloon, the flow passage walls are compressed to increase the mean cross sectional diameter of the flow passage.

In an exemplary embodiment, at least a portion of the implant comprises a hollow chamber, for example, adapted to be inflated. Optionally, the hollow chamber is adapted to assume multiple sizes, for example using varied inflation pressures, thereby providing different effective cross-sectional diameters of the flow passage.

In an exemplary embodiment, the axis of the outer surface is non-parallel to the longitudinal axis of the flow passage so that optionally the outer surface configuration conforms to the shape of the blood vessel where the implant is located.

An aspect of some embodiments of the invention relates to a flow-obstructing implant adapted for implantation in a blood vessel, having a wall that defines a flow passage and one or more flaps projecting from the wall into the flow passage. Optionally, the one or more flaps may be angularly adjusted with respect to the flow passage, thereby adjusting the flow of blood through the flow passage. In an exemplary embodiment, angular adjustment of the flap position with respect to the flow passage is made using an inflatable balloon, for example an inflatable portion of a balloon catheter.

An aspect of some embodiments of the invention relates to a flow-obstructing implant having two or more flow obstructing flaps projecting therefrom, wherein two or more of the flaps are connected by at least one guide element. In the expanded state, the at least one guide element is operative to encourage the two or more flaps into a position in which they partially block the flow passage.

Optionally, the two or more flaps connected to the guide element comprise shape memory materials that assume a final stable expanded position so that the guide elements are no longer necessary for position encouragement. In an exemplary embodiment, the one or more guide elements may comprise materials that sever, and/or expand, during adjustment of flap position for example using a balloon catheter.

An aspect of some embodiments of the invention relates to a flow-obstructing implant adapted for implantation in a blood vessel having a wall that defines a flow passage and at least one wire projecting from the wall. In an exemplary embodiment, at least a portion of the at least one wire comprises a width that at least partially obstructs blood flow through the passage. Optionally, the at least one wire comprises a hollow tube that, for example, is inflatable. Optionally, the at least one wire comprises a varying effective width.

Optionally, the at least one wire comprises at least two wires, for example that are interconnected. In an exemplary embodiment, the two or more wires are connected to a curved junction, for example a plate with curved edges, for the purpose of reducing turbulence in blood flow. Optionally the two or more wires incorporate a substantially volumetric object, for example a sphere.

There is thus provided a tubular implant for obstructing blood flow through a blood vessel, the implant comprising an outer surface having a geometry of a tube, at least a portion of which is adapted for contacting a blood vessel and an inner surface defining a passage through which blood flows, wherein the distance between the inner surface and the outer surface is non-uniform along an axis of the tube.

In an exemplary embodiment, at least a portion of the inner and outer walls are continuous. Further, at least one portion of the distance is hollow. Optionally, the at least one hollow portion is adapted to be inflated.

In an exemplary embodiment, at least one of the outer and inner surfaces is parallel to the longitudinal axis of the flow passage. Optionally, at least one of the outer and inner surfaces is non-parallel to the longitudinal axis of the flow passage.

There is thus further provided an implant for obstructing blood flow in a blood vessel, the implant comprising a tubular wall defining a flow passage adapted for encircling a flow of blood through a vessel and one or more positionally adjustable flaps projecting from the wall into the blood flow. In an exemplary embodiment, the one or more flaps comprise two or more flaps.

There is thus further provided an implant for obstructing blood flow in a blood vessel, the implant comprising a tubular wall defining a flow passage adapted for encircling a flow of blood through a vessel two or more positionally adjustable flaps each connected at one end to the tubular wall and one or more guide elements connecting the two or more flaps, operative to maintain the two or more flaps in a position in which they partially block the flow passage.

Optionally, the one or more guide elements deform or break under pressure. Alternatively the one or more guide elements comprise two or more guide elements. Optionally, the two or more guide elements have different pressure thresholds at which they deform or break.

There is thus further provided an implant for obstructing blood flow in a blood vessel, the implant comprising a tubular wall defining a flow passage adapted for encircling a flow of blood through a vessel and at least one non-overlapping flap projecting from the wall into the blood flow.

In an exemplary embodiment, the at least one flap is substantially planar with a surface of the tubular wall. Optionally, the at least one flap is substantially non-planar with a surface of the tubular wall. Alternatively or additionally the at least one flap is positionally adjustable.

In an exemplary embodiment, the at least one flap comprises at least two non-overlapping flaps. Optionally, the implant comprises a kit that additionally includes a flap angle adjusting tool, the tool comprising a shaft having one or more wing projections adapted to press against one or more flow obstructing flaps. Optionally, the one or more wings of the tool are activated in one or both of mechanically and inflatably.

There is thus further provided an implant for obstructing blood flow in a blood vessel, the implant comprising a tubular wall defining a flow passage adapted for encircling a flow of blood through a vessel and least one wire of varying effective width adapted to at least partially obstruct blood flow.

Optionally, the at least one wire curves in a plane of the width of the wire. Alternatively or additionally, the at least one wire is connected to an object. Alternatively or additionally, the at least one wire comprises at least two wires. Optionally, the at least two wires are interconnected, for example, the interconnection comprises at least one curved member.

In an exemplary embodiment, at least a portion of the implant is adapted to change configuration upon absorption of fluid. Alternatively or additionally, at least a portion of the implant comprises resilient materials.

In an exemplary embodiment, at least a portion of the implant comprises shape memory materials. Alternatively or additionally at least a portion of the implant is adapted to be inflated.

There is thus provided a method of modifying an implant geometry, of a tubular implant with at least one intra-luminal flap, comprising contacting at least one intra-lumen flap of an implanted vascular implant with an effector element and bending the flap by applying force via the contact. Optionally, contacting comprises pulling the element towards the flap. Alternatively or additionally, contacting comprises pushing the element towards the flap.

In an exemplary embodiment, pushing comprises pushing with enough force to tear an element restraining of the flap. Optionally, the element comprises a mechanically expandable element. alternatively or additionally the element comprises a mechanically expandable element.

There is thus provided an implant comprising a radially expandable tubular sheath and at least one flap welded to the sheath and configured to at least partially and rigidly obstruct a lumen of the sheath. Optionally, the tubular sheath comprises a wire mesh sheath. In an exemplary embodiment, the implant comprises at least two flaps and comprising at least one restraining element interconnecting the flaps and limiting their movement relative to each other. Optionally, the restraining element is adapted to be torn by applying force to one or more flaps, while implanted.

### BRIEF DESCRIPTION OF THE DRAWINGS

Exemplary non-limiting embodiments of the invention are described in the following description, read with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a longitudinal cross section of a flow-obstructing implant installed in a blood vessel, in accordance with an exemplary embodiment of the invention;

FIGS. 2A and 2B are isometric views of two embodiments of flow-obstructing implants with flaps, in accordance with an exemplary embodiment of the invention;

FIGS. 3A-3D are various embodiments of flow-obstructing implants having narrowed passages, in accordance with an exemplary embodiment of the invention;

FIG. 4A-4C are various embodiments of flow-obstructing implants having wires, in accordance with an exemplary embodiment of the invention;

FIGS. 5A and 5B are implants with guide elements spanning the flow obstructing flaps, in accordance with an exemplary embodiment of the invention;

FIGS. 6A-6D are an embodiment and operation of a tool that adjusts the angle of flow-obstructing flaps, in accordance with an exemplary embodiment of the invention; and

FIGS. 7A-7C are an alternative embodiment and operation of a tool that adjusts the angle of flow-obstructing flaps, in accordance with an exemplary embodiment of the invention.

### DETAILED DESCRIPTION OF EXEMPLARY EMBODIMENTS

Thick-Walled Implant

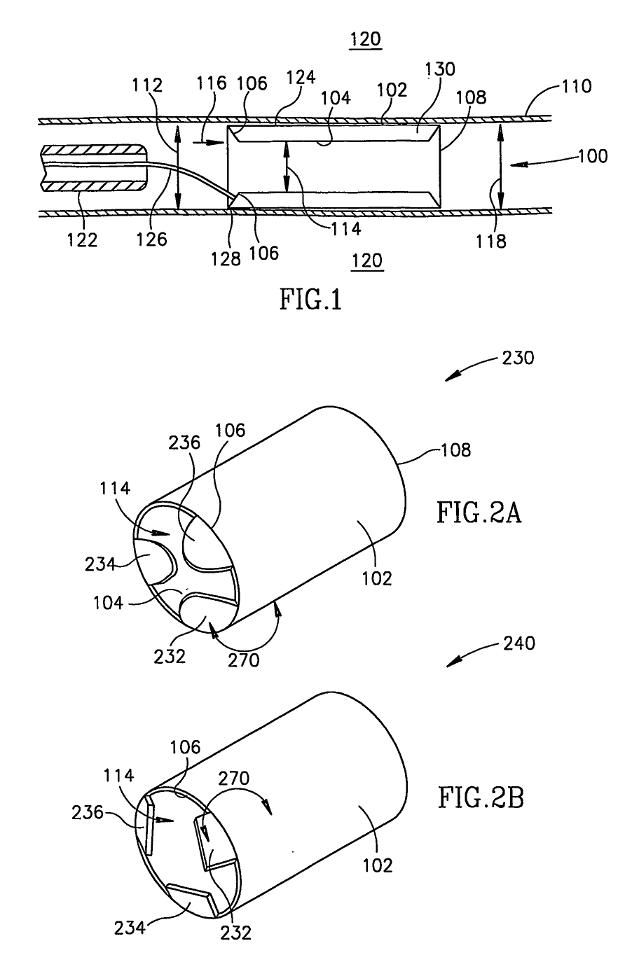

FIG. 1 is a longitudinal section of a flow-obstructing implant 100 installed in a blood vessel 110, comprising an outer wall 102 and an inner wall 104 and a cylindrical ring 130 comprising solid material between walls 102 and 104. In an exemplary embodiment, inner wall 104 defines a lumen 114 that is narrower in diameter than a blood vessel pre-implant diameter 112, thereby reducing blood volume in a post-implant area 118 as blood flows in a direction 116.

In an exemplary embodiment, implant 100 is implanted in a coronary vein and the reduction of blood flow promotes angiogenesis in an area of coronary tissue 120. Further details of angiogenesis are provided in “The Surgical Management of Coronary Artery Disease: Background, Rationale, Clinical Experience” by C. S. Beck and B. L. Brofman, American College of Physicians in Annals of Internal Medicine Vol. 45, No. 6, December 1956.

Alternatively or additionally, implant 100 is implanted in other vessels, for example arteries, the coronary sinus, portal vein, hepatic and/or other veins.

In an exemplary embodiment, inner wall 104, chamber ring 130 and/or outer wall 102 comprise shape memory materials that automatically expand when released from a compressive force. Implant 100, for example, is delivered to the deployment site in blood vessel 110 in a compressed size inside a delivery catheter 122. Upon reaching the in situ area, implant 100 is freed of delivery catheter 122 and expands automatically.

Alternatively or additionally, inner wall 104, ring 130 and/or outer wall 102 comprise materials that absorb liquid, for example, from the blood flowing through blood vessel 110 and change size and/or configuration as a result of the absorption. In an exemplary embodiment, implant 100 is delivered in a compressed state to the delivery site, freed of catheter 122 and absorbs liquid to expand into its final configuration.

Optionally, at least a portion of wall 104 comprises a material that can be compressed and/or deformed under pressure. A balloon catheter, for example, is expanded in lumen 114, thereby increasing the flow passage.

In an exemplary embodiment, walls 102 and 104 comprise a flexible material and ring 130 comprises an inflatable area (e.g. a hollow chamber). To inflate ring 130, fluid is pumped into ring 130 using inflator hose 126. Upon completion of inflation, inflator hose 126 is pulled free of implant 100 and an inflator seal 128 automatically seals implant 100. Optionally, chamber 126 can be inflated to two or more sizes, thereby providing variably obstruction to blood flow.

In an exemplary embodiment, inflator hose 126 is left in place for a period of time, for example 24 hours, during which the changes in blood flow volume, pressure and/or other factors are measured. Considering these measurements, implant 100 is inflated and/or deflated to provide to achieve a desired obstruction.

Implant Having Flaps

FIGS. 2A and 2B are isometric views of obstructing implants 230 and 240 comprising three non-overlapping flaps 232, 234 and 236 that allow blood flow, for example, between their adjacent borders. Optionally, flaps 232, 234 and 236 are configured without sharp edges along their borders projecting into the blood flow so that turbulence in blood flow is minimized.

Implant 240 comprises flaps that are skewed in relation to outer wall 102. The skewed relationship of implant 240 allows the extents of flaps 232, 234 and 236 around an axis running through lumen 114 to be enlarged to a maximal extent without overlap between the flaps. Implant 230 comprises flaps 232, 234 and 236 that are not skewed in relation to outer wall 102 but the angle governing their projection into lumen 114 may be adjusted.

In an exemplary embodiment, at least one flap 232 is adjustable in an angle 270 with respect to implant 230 or 240. For example, following implantation of implant 230 or 240, a balloon catheter is placed in lumen 114 and inflated so that it presses against flap 232. As the balloon is inflated, angle 270 decreases and flap 232 provides less obstruction to blood flowing through lumen 114. Alternatively or additionally, changing the angle of flap 232 encourages the walls of the surrounding vessel 110 (FIG. 1) to collapse around the flaps, providing better anchoring of implant 230.

In an exemplary embodiment, by inflating a balloon catheter adjacent flap 232, the skew angle of flap 232 in implant 240 is adjusted, for example encouraging anchoring in vessel 110.

Optionally, changes in the flow of blood during adjustment of the position of flap 232 are measured, for example using an angiogram, and positional adjustment of flap 232 is made until an appropriate blood flow is achieved. For further details on achieving proper blood flow obstruction, see “Implant Installation Technique”, below.

Alternatively or additionally, a balloon catheter is moved in direction 116 (FIG. 1) until it presses against the front of flap 232. As the balloon is inflated, flap 232 is pushed into lumen 114. As angle 270 increases, the flow of blood through lumen 114 is reduced. Optionally, flaps 232, 234 and 236 are interconnected with a flexible membrane that increases the obstruction area of the flaps. As flaps 232, 234 and 236 move, the membrane expands or contracts.

Flaps with Restraints

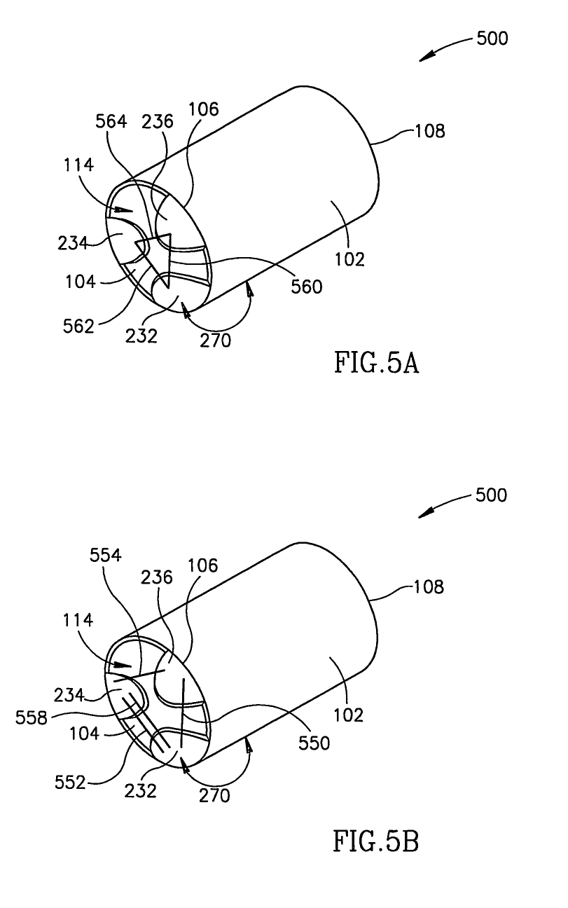

FIG. 5A is an embodiment of a flow-obstructing implant 500 having:
flaps 232, 234 connected by a guide element 562;
flaps 234, 236 connected by a guide element 564; and
flaps 236, 232 connected by a guide element 560.

In an exemplary embodiment, guide elements 560, 562 and 564 are positioned substantially close to the edges of flaps 232, 234 and 236 that are closest to the center of lumen 114.

Guide element 562, for example, cause flaps 232 and 234 to offset from wall 104, and project into lumen 114 when implant 500 is expanded in situ. Alternatively or additionally when flaps 232 and 234 extend beyond front edge 106 then when implant 500 is expanded, guide element 562 cause flaps 232 and 234 to be at an angle to the radial axis of implant 500.

Optionally, flaps 232 and 234 are configured from a shape memory material so that following expansion of implant 500, attachment to guide element 562 becomes unnecessary.

Optionally, the angle of 232 and 234 may be adjusted using an adjusting tool 600 or 700, as described below and guide element 562 comprises a material that severs and/or expands under pressure. In such embodiments, during adjustment of the angle of flaps 232 and 234, guide element 562 is severed or stretched. Alternatively or additionally guide element 562 may comprise a biologically dissolvable material that dissolves in vivo over a period of time.

FIG. 5B is an embodiment of a flow-obstructing implant 500 having:
flaps 232, 234 connected by a guide element 552;
flaps 234, 236 connected by a guide element 554; and
flaps 236, 232 connected by a guide element 550.

In an exemplary embodiment, guide element 550 is positioned relatively close to front end 106, reducing its size over guide element 560. By reducing the size of element 550, blood turbulence may be reduced.

Optionally, flaps 232 and 234 may be connected by two guide elements, 552 and 558. Elements 552 and 558 optionally sever and/or expand at different pressures during adjustment of flap angle. Alternatively or additionally, elements 552 and 558 are of different lengths. In an exemplary embodiment, when a balloon catheter is inflated to a first circumference, flaps 232 and 234 move outward and guide element 558 expands and/or severs so that flaps 232 and 234 maintain a first expanded circumference with respect to wall 102.

In an exemplary embodiment, a balloon catheter is inflated to a second circumference, flaps 232 and 234 move outward to a second expanded position and guide element 552 expands and/or severs. With both guide elements 552 and 558 expanded and/or severed, flaps 232 and 234 assume a second expanded circumference with respect to wall 102.

Angle Adjusting Tool

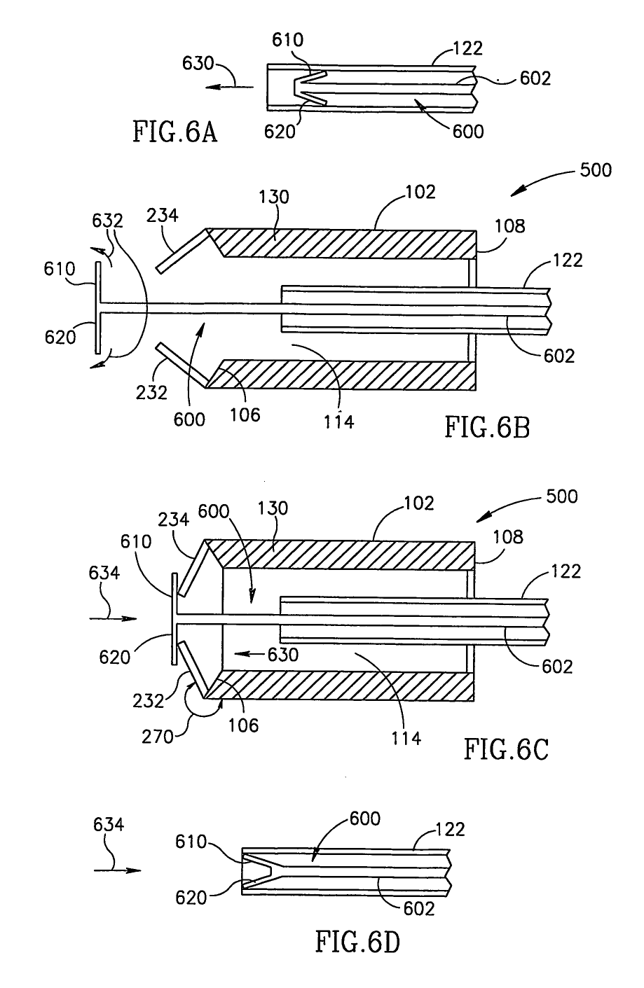

FIGS. 6A-6D show use of flap angle adjusting tool 600, for example included in a kit together with implant 500. Adjusting tool 600 comprises a hollow tubular shaft 602 connected to inflatable wings 610 and 620. In an exemplary embodiment, adjusting tool 600 is transported in delivery catheter 122 with wings 610 and 620 retracted, as seen in FIG. 6A. Upon reaching implant 500, adjusting tool 600 is pushed forward in a direction 630 until wings 610 and 620 are beyond catheter 122.

In FIG. 6B, a fluid passes through tubular shaft 602 and causes wings 610 and 620 to open (moving in a direction 632) so they project radial outward of the axis of shaft 602. As seen in FIG. 6C, adjusting tool 600 is pulled in a direction 634 so that wings 610 and 620 press against flaps 232 and 234 causing angle 270 to increase, thereby increasing obstruction of blood flow.

Alternatively or additionally, wings 610 and 620 may be positioned in lumen 114 and pressed in a direction 630 against flaps 232 and 234, causing angle 270 to decrease, thereby reducing blood flow obstruction.

In FIG. 6D, collapse of tool 600 is shown. Wings 610 and 620 have been made non-rigid, for example by removing fluid from wings 610 and 620 via tube 602. Adjusting tool 600 is then pulled in a direction 634, causing wings 610 and 620 to extend beyond shaft 602 as tool 600 is pulled into delivery catheter 122.

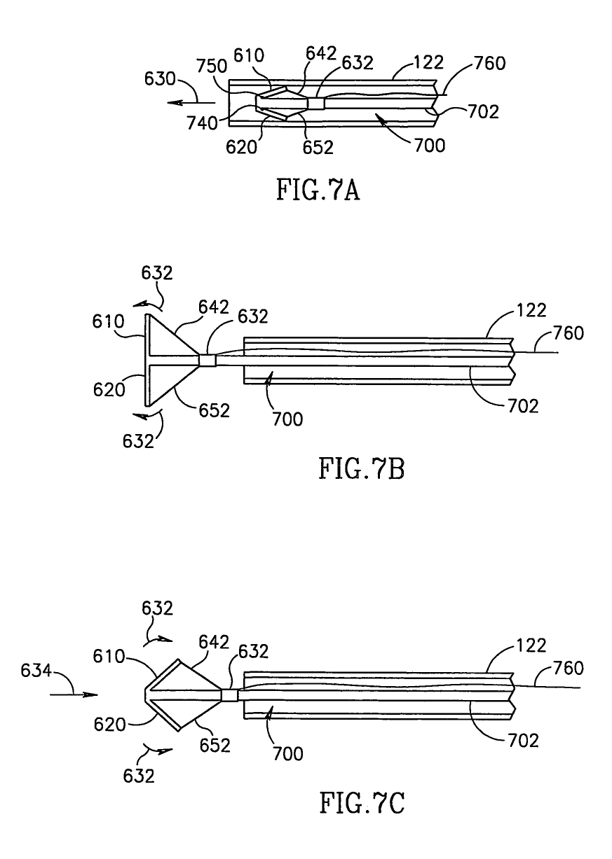

FIGS. 7A-7C show use of an alternative embodiment of an adjusting tool 700 that is activated mechanically. In an exemplary embodiment, wings 610 and 620 are rotatably attached to a shaft 702, for example with spring hinges 740 and 750. Adjusting tool 700 is transported in catheter 122 and moved in direction 630 so that it is beyond catheter 122 allowing spring hinges 740 and 750 to cause wings 610 and 620 to expand radially outward in direction 632 (FIG. 7B).

With wings 610 and 620 in the expanded position, adjusting tool 700 is used to modify the position of flaps 232 and 234. Optionally, this can be accomplished either by pulling tool 700 in direction 634 against the forward aspect of flaps 232 and 234. Alternatively tool 700 may be pushed in direction 630 against the lumen-facing surfaces of flaps 232 and 234.

Optionally, removal of tool 700 is accomplished by pulling tool 700 in direction 634 into catheter 122, causing wings 610 and 620 to extend beyond shaft 702 (similar to the position of adjusting tool 600 in FIG. 6D). In an exemplary embodiment, the pressure required to cause the collapse of wings 610 and 620 is greater than the pressure exerted during adjustment of the angle of flap 232 so that wings 610 and 620 do not inadvertently collapse during the adjustment.

In an alternative exemplary embodiment, wings 610 and 620 are connected to a collar 632 by struts 642 and 652 and collar 632 is connected to a user-operated wire 760. By pulling wire 760 in direction 634 with respect to shaft 702, collar 632 moves in direction 634, so that wings 610 and 620 collapse in a direction 732 against shaft 702.

Adjusting tool 700 with collapsed wings 610 and 620 is pulled into catheter 122 and removed from the vicinity of implant 500 (FIG. 6C) and out of the patient.

Narrow Passage Implant

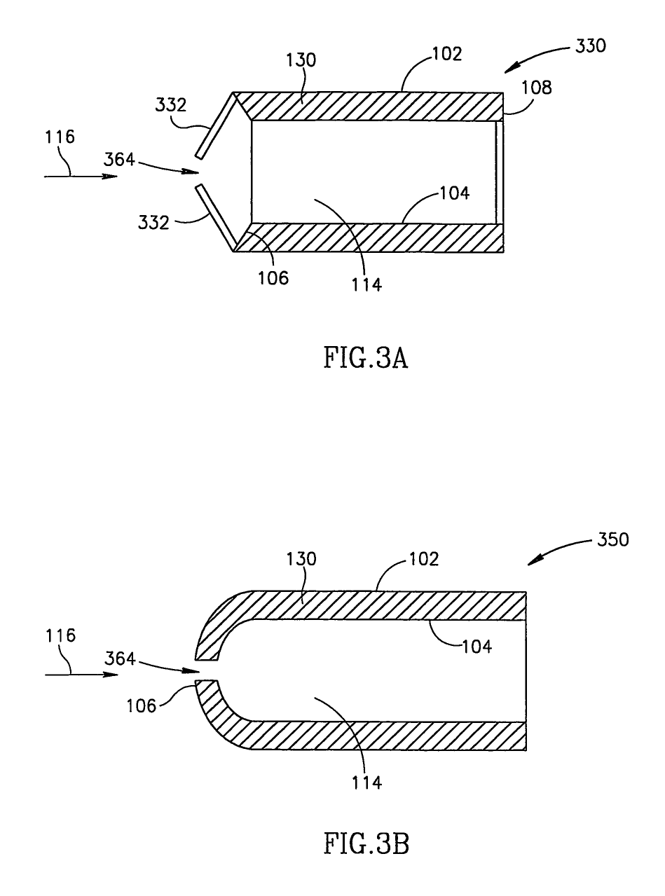

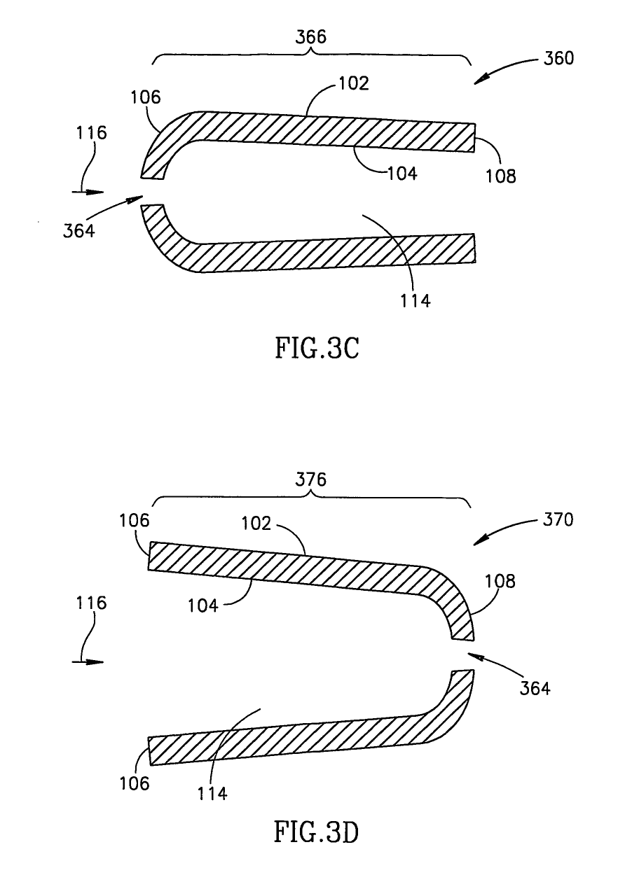

FIGS. 3A-3D show various embodiments of implants 330, 350, 360 and 370, having a narrow opening 364 that obstructs blood flow rather than, for example, individual flaps 232 of implant 230. In an exemplary embodiment, the material surrounding passage 364 is flexible so that passage 364 can expand under pressure. In an exemplary adjustment procedure, a balloon is inflated in passage 364, thereby causing expansion of the flexible material so that passage 364 increases in diameter.

The various embodiments of implants 330 may have specific designs for use in a specific blood vessel environment For example implant 370 (FIG. 3D) that has a tapered section 376 may be suitable for use in a tapered blood vessel.

Implant 330, 340, 350 and 360 have front walls 106 that curve toward opening 364 into lumen 114, for example encouraging the blood vessel to collapse around the implant so that it doesn't shift following implantation.

Implant 360 demonstrates a tapered section 366 that reduces the internal volume of passage 114 along flow path 116 possibly enhancing the angiogenic affect by causing pooling of blood after it passes through opening 364.

In some cases, pooling of blood inside lumen 114 is desired to enhance angiogenesis. To this end, implant 360 may be reversed in its implantation in a blood vessel so that blood in lumen 114 causes increased backflow pressure as the blood flow is obstructed from passing through (exit) opening 364.

Implant 370 demonstrates tapered section 376 and has its opening 264 at end 108 with respect to blood flow 116 that similarly increase pooling of blood in lumen 114. Angiogenesis may be increased by any combination of increased pressure, pooling and backflow of blood.

Implant 330 shows a front wall 332 having a difference thickness and/or comprising a different material than wall 102 and/or ring 130. In an exemplary embodiment, front wall 332 comprises a machined surface that encourages tissue ingrowth, thereby promoting implant 330 to anchor in the blood vessel.

Optionally, front wall 332 comprises a shape memory material that folds or compresses to fit inside catheter 122 (FIG. 1). Walls 102 and 104 optionally comprise a material with resilient properties. Upon release from catheter 122, wall 332 unfolds and assumes its implanted shape, encouraging resilient walls 102 and/or 104 to assume their implanted configuration.

Shape memory materials may include, stainless steel mesh, surgical grade titanium and/or other metals. Alternatively or additionally, implant 330, including walls 102 and 104, may comprise a resilient material having a jacket of steel mesh surrounding outer wall 102. In an exemplary embodiment, the mesh jacket provides a surface that enhance anchorage into blood vessel 110 (FIG. 1).

Implant Having Wires

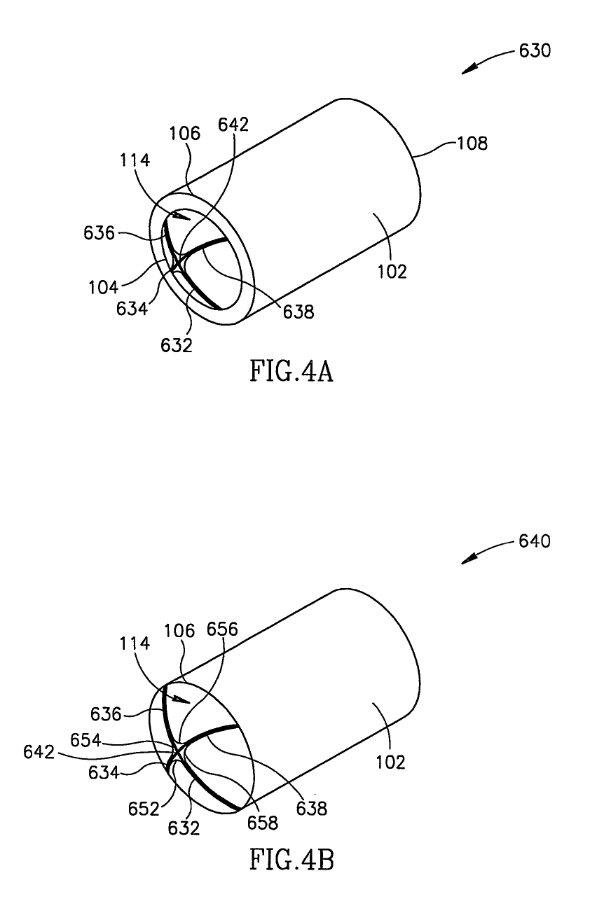

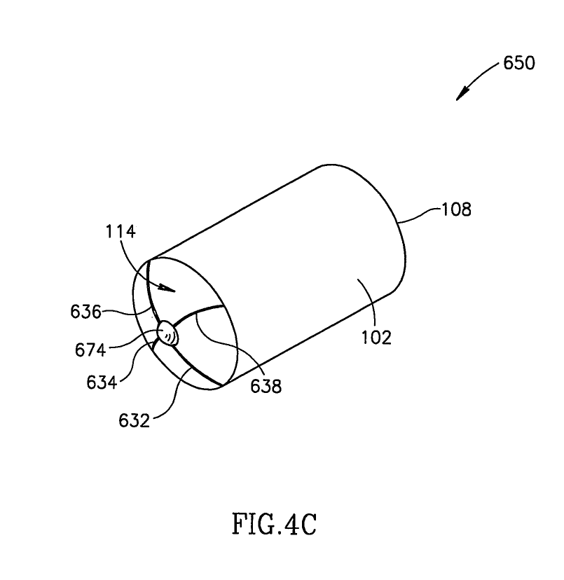

FIGS. 4A-4C are isometric views of implants 630, 640 and 650, comprising at least one flow obstructing wire 232 that curves in the plane of the width of wire 632.

In exemplary embodiments, as shown in implants 630 and 640, at least one wire 632 is connected to a plate 642. Optionally, flow obstructing wire 232 comprises four wires, 632, 634, 636 and/or 638 that are connected to plate 642. In an exemplary embodiment, plate 642 provides obstruction of blood flow. Alternatively or additionally plate 642 may comprise an open ring that serves as a junction of wires 632, 634, 636 and/or 638 to minimize turbulence of blood flow that may occur when the wires are joined without a ring to which they are connected.

Alternatively or additionally, wires 632, 634, 636 and/or 638 comprises flat ribbon-like elements, for example, that have varying effective width when laid out in a flat plane.

In implant 650, at least one wire 632 is connected to a volumetric object, for example a sphere 674.

In an exemplary embodiment, the cross-sectional shape of sphere 674 and/or plate 642 may comprise any one of a variety of sizes and/or shapes for example flat spheroid, triangular or square. These and other shapes of sphere 674 and/or plate 642 may be chosen, based upon the amount of flow obstruction required and/or turbulence (or lack of turbulence) desired.

Optionally, plate 642, sphere 674 and/or wire 632 comprise a material that expands upon absorbing a liquid. Alternatively or additionally, sphere 674, wire 632 and/or wall 102 are inflatable and implant 650 is inflated, for example, using inflator hose 126 (FIG. 1).

Implant 640 shows details of plate 642 that comprises curvatures 652, 654, 656 and 658 that smooth the interconnection between the wires and plate 642, thereby reducing blood turbulence. Alternatively or additionally plate 642 and/or sphere 674 may not be centered with respect to lumen 114 and/or may comprise more than one plate 642 and/or sphere 674.

Implant 630 is shown with a front end 106 being thickened with respect to a rear end 108 of implant 640, thereby adding to the obstruction of blood flow. Front wall 106 is shown as being planar and perpendicular to wall 102. In an alternative embodiment, wall 106 is sloped into lumen 114 or may have a curved surface. The thickness and/or configuration of wall 106, may be influenced by a variety of factors including the blood pressure and/or the thickness of the blood vessel walls.

Wire Construction

For simplicity, reference will be made to construction of single wire 632, though such references could apply to wires 634, 636 and/or 638 as well. In an exemplary embodiment, wire 632 is resilient so that it folds into a compressed state while implant 630 is compressed within delivery catheter 122. Optionally, resilient wire 632 automatically forms into a pre-determined configuration shape upon exiting catheter 122 (FIG. 1), for example independent of the expansion of wall 102.

In an exemplary embodiment, wire 632 comprises flexible material whose shape, for example, is determined by the amount of drag in the blood flowing around it. In an exemplary embodiment, wire 632 moves according to changes in blood flow and/or blood pressure during the cardiac cycle.

Wire 632 is shown at front end 106 though it could be located anywhere along lumen 114, including rear end 108. Wire 632 is shown projecting forward of front end 106, though it could be perpendicular to outer wall 102 or even project into lumen 114, for example as a result of blood flowing into lumen 114.

Optionally, wire 232 comprises a tube that has a varying effective width and may, for example, be altered by inflation or deflation. In an exemplary embodiment, for example wire tube 232 has a fixed narrow attachment to plate 634 while the remainder of wire tube 232 has an effective diameter that increases in response to inflation. Inflation of wire tube 232 initially may result in a tube that of uniform effective diameter while increased inflation may cause an increase in effective width of at least a portion of wire tube 232 beyond the area of its attachment to plate 634.

In an exemplary embodiment, wire 632 tube inflates to and/or comprises a width of between 0.1-1 millimeters (optionally less than 0.1 millimeters or more than 1 millimeter) to provide obstruction of blood flow.

In an exemplary embodiment, plate 642 has an area of between 0.5 and 1.0 square millimeters (optionally less than 0.5 square millimeters or more than 1 square millimeter) to provide obstruction of blood flow. In an exemplary embodiment, sphere 674 comprises a volume of between 0.1-1 cubic millimeters (optionally less than 0.1 cubic millimeters or more than 1 cubic millimeter) to provide obstruction of blood flow.

Further changes in effective area of wire 632, sphere 674 and/or plate 642 are contemplated for the purpose of modifying the blood flow obstruction.

Implant Materials

In an exemplary embodiment of the invention, implant 100 is cut out of a sheet of metal or a tube, for example, using laser, water cutting, chemical erosion or metal stamping (e.g., with the result being welded to form a tube). Alternatively or additionally one or more of flaps 232 are welded to surface 104 or edge 108 or 106 of implant 100. In an exemplary embodiment, as implant 100 expands, for example during implantation, the distance between flaps 232, 234 and 236 increases or decreases based upon the amount of expansion of implant 100.

Alternatively or additionally, implant 100 is woven (e.g., of metal or plastic fiber), for example, using methods well known in the art.

In an exemplary embodiment of the invention, implant 100 is formed of metal, for example, a NiTi alloy (e.g., Nitinol) or stainless steel (e.g., 316L and 316LS). Alternatively, implant 100 is formed of, or coated with, other biocompatible materials, such as nylon and/or other plastics. Optionally, implant 100 is formed of two or more materials, for example, inner wall 104 being formed of plastic and outer wall 102 being formed of metal.

Optionally, an outer surface 124 (FIG. 1) is manufactured with a machining process and, for example, etched in a pattern on at least a portion of an outer surface 124, so that it anchors against blood vessel 110. Alternatively or additionally, outer surface 124 is fashioned with knobs and/or indentations that promote ingrowth of tissue 120 that aid in anchoring implant 100. Alternatively or additionally, the diameter of outer wall 102 may be varied along its length to conform to contact a portion of blood vessel 110 when blood vessel 110 has, for example, a variable configuration and/or diameter along its length.

In embodiments including inflatable ring 130, implant 100 may comprises flexible materials, for example silicone. Alternatively or additionally, implant 100 may comprise embodiments that enhance anchoring in vessel 110 (FIG. 1). For example, along opening 108 and/or 104, serrations may be provided that enhance anchoring into vessel 110. Alternatively or additionally, wall 102 may be roughened to enhance anchoring. Providing serration and/or roughening to implant outer wall 102, for example, may be accomplished by any one of a variety of methods known in the art, some of which are detailed below.

In an exemplary embodiment, implant 100 comprises materials that prevent coagulation, embolism formation and/or bacterial colonization and, are released over a period of time. The time release of the materials may be set in advance so that release occurs over a period of one month or more or two weeks or less, depending, for example on the patient state of health.

Determining Implant Size

In an exemplary procedure used in an embodiment of the present invention, an angiogram is made that includes the flow through blood vessel 110. The shape and/or cross sectional diameters of blood vessel 110 are determined from the angiogram and an implant 100 having an appropriate size, shape and/or configuration is chosen to be implanted.

For example, the outside diameter and configuration of implant 100 are matched to the inside diameter and configuration of blood vessel 110 to provide an optimal fit with blood vessel 110. Further, the cross sectional configuration of lumen 114, for example, is matched to the profile of obstruction determined to provide the best results.

Alternatively or additionally, once implant 100 is in place, an angiogram of blood vessel 110 is made and one or more changes are made to change blood flow through passage 114, for example, using a balloon catheter. Changes in implant 100 may be accomplished, for example by inflating a balloon in lumen 114 and/or in proximity to front end 106 as noted above. Adjustment of implant 100 may affect one or more of:
walls 102 and 104;
flaps 232, 234 and 236;
wires 632, 634, 636 and/or 638;
ring 130; and
lumen 114.

In an exemplary embodiment, a desired change in the blood volume is accomplished by volumetric measurements. For example, to achieve a 50% reduction in blood flow, the cross sectional diameter of blood vessel 110 is determined from the angiogram. In an exemplary embodiment, implant 100 is manufactured with different diameters of lumen 114 and an implant 100 with an appropriate diameter of narrow lumen 114 is chosen to make this reduction.

Alternatively or additionally, the thickness of ring 130, outer wall 102 and/or inner wall 104 are chosen in order to reduce blood flow to a specific level, regardless of the percentage change of flow reduction.

It should be appreciated that different features may be combined in different ways. In particular, not all the features shown above in a particular embodiment are necessary in every similar exemplary embodiment of the invention. Further, combinations of features from different embodiments into a single embodiment or a single feature are also considered to be within the scope of some exemplary embodiments of the invention.

In addition, some of the features of the invention described herein may be adapted for use with prior art devices, in accordance with other exemplary embodiments of the invention. The particular geometric forms and measurements used to illustrate the invention should not be considered limiting the invention in its broadest aspect to only those forms. Although some limitations are described only as method or apparatus limitations, the scope of the invention also includes apparatus designed to carry out the methods and methods of using the apparatus.

Also within the scope of the invention are surgical kits, for example, kits that include sets of delivery systems and implants. Optionally, such kits also include instructions for use. Measurements are provided to serve only as exemplary measurements for particular cases, the exact measurements applied will vary depending on the application. When used in the disclosure and/or claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A tubular implant for obstructing blood flow through a blood vessel, the implant comprising:
an outer surface having a geometry of a tube, at least a portion of which is adapted for contacting a blood vessel; and
an inner surface defining a passage through which blood flows, wherein the distance between the inner surface and the outer surface is non-uniform along an axis of the tube.

2. An implant according to claim 1, wherein at least a portion of the inner and outer walls are continuous.

3. An implant according to claim 1, wherein at least one portion of the distance is hollow.

4. An implant according to claim 3, wherein the at least one hollow portion is adapted to be inflated.

5. An implant according to claim 3, wherein at least one of the outer and inner surfaces is parallel to the longitudinal axis of the flow passage.

6. An implant according to claim 3, wherein at least one of the outer and inner surfaces is non-parallel to the longitudinal axis of the flow passage.

7. An implant for obstructing blood flow in a blood vessel, the implant comprising:
a tubular wall defining a flow passage adapted for encircling a flow of blood through a vessel; and
one or more positionally adjustable flaps projecting from the wall into the blood flow.

8. An implant according to claim 7, wherein the one or more flaps comprise two or more flaps.

9. An implant for obstructing blood flow in a blood vessel, the implant comprising:
a tubular wall defining a flow passage adapted for encircling a flow of blood through a vessel;
two or more positionally adjustable flaps each connected at one end to the tubular wall; and
one or more guide elements connecting the two or more flaps, operative to maintain the two or more flaps in a position in which they partially block the flow passage.

10. The implant according to claim 9 wherein the one or more guide elements deform or break under pressure.

11. The implant according to claim 9, wherein the one or more guide elements comprise two or more guide elements.

12. The implant according to claim 11 wherein the two or more guide elements have different pressure thresholds at which they deform or break.

13. An implant for obstructing blood flow in a blood vessel, the implant comprising:
a tubular wall defining a flow passage adapted for encircling a flow of blood through a vessel; and
at least one non-overlapping flap projecting from the wall into the blood flow.

14. An implant according to claim 13, wherein the at least one flap is substantially planar with a surface of the tubular wall.

15. An implant according to claim 13, wherein the at least one flap is substantially non-planar with a surface of the tubular wall.

16. An implant according to claim 13, wherein the at least one flap is positionally adjustable.

17. An implant according to claim 13, wherein the at least one flap comprises at least two non-overlapping flaps.

18. An implant according to claim 13, comprising a kit that additionally includes a flap angle adjusting tool, the tool comprising a shaft having one or more wing projections adapted to press against one or more flow obstructing flaps.

19. The implant according to claim 18, wherein the one or more wings of the tool are activated in one or both of the following ways:
mechanically; and
inflatably.

20. An implant for obstructing blood flow in a blood vessel, the implant comprising:
a tubular wall defining a flow passage adapted for encircling a flow of blood through a vessel and least one wire of varying effective width adapted to at least partially obstruct blood flow.

21. An implant according to claim 20, wherein the at least one wire curves in a plane of the width of the wire.

22. An implant according to claim 20, wherein the at least one wire is connected to an object.

23. An implant according to claim 20, wherein the at least one wire comprises at least two wires.

24. An implant according to claim 23, wherein the at least two wires are interconnected.

25. An implant according to claim 24, wherein the interconnection comprises at least one curved member.

26. An implant according to claim 1, wherein at least a portion of the implant is adapted to change configuration upon absorption of fluid.

27. An implant according to claim 1, wherein at least a portion of the implant comprises resilient materials.

28. An implant according to claim 1, wherein at least a portion of the implant comprises shape memory materials.

29. An implant according to claim 1, wherein at least a portion of the implant is adapted to be inflated.

30. A method of modifying an implant geometry, of a tubular implant with at least one intra-luminal flap, comprising:
contacting at least one intra-lumen flap of an implanted vascular implant with an effector element; and
bending said flap by applying force via said contact.

31. A method according to claim 30, wherein contacting comprises pulling said element towards said flap.

32. A method according to claim 30, wherein contacting comprises pushing said element towards said flap.

33. A method according to claim 32, wherein pushing comprises pushing with enough force to tear an element restraining of said flap.

34. A method according to claim 30, wherein said element comprises a mechanically expandable element.

35. A method according to claim 30, wherein said element comprises a mechanically expandable element.

36. An implant comprising:
a radially expandable tubular sheath; and
at least one flap welded to said sheath and configured to at least partially and rigidly obstruct a lumen of said sheath.

37. An implant according to claim 36, wherein said tubular sheath comprises a wire mesh sheath.

38. An implant according to claim 36, comprising at least two flaps and comprising at least one restraining element interconnecting said flaps and limiting their movement relative to each other.

39. An implant according to claim 38, wherein said restraining element is adapted to be torn by applying force to one or more flaps, while implanted.
